(19) United States
(12) Patent Application Publication
(10) Pub. No.: US 2021/0085216 A1
(43) Pub. Date: Mar. 25, 2021

(54) Wearable Health Device System with Normalized Seismocardiography Signals

(71) Applicant: Robert Bosch GmbH, Stuttgart (DE)

(72) Inventors: Christian Peters, Sunnyvale, CA (US); Thomas Rocznik, Mountain View, CA (US); Seow Yuen Yee, Mountain View, CA (US); Robert Duerichen, Oxford (GB); Vincent-Johannes Schnitzbauer, Wäschenbeuren (DE)

(21) Appl. No.: 16/975,010

(22) PCT Filed: Feb. 25, 2019
(86) PCT No.: PCT/EP2019/054549

§ 371 Date: Aug. 21, 2020

Related U.S. Application Data
(60) Provisional application No. 62/635,183, filed on Feb. 26, 2018.

(51) Int. Cl. A61B 5/11; A61B 5/00; G16H 40/67; G16H 50/70; G16H 10/65; G16H 50/30; G16H 50/20

(52) U.S. Cl. CPC A61B 5/1102; A61B 5/489; A61B 5/7246; A61B 5/7214; A61B 5/7278; G16H 40/67; A61B 5/02438; G16H 10/65; G16H 50/30; G16H 50/20; A61B 2562/0219; A61B 5/0004; G16H 50/70

(57) ABSTRACT

A wearable health device system includes a housing configured to be worn by a subject, and a sensor assembly with at least two accelerometers which sense acceleration along non-parallel axes. A processor operably connected to the sensor assembly and a memory executes program instructions in the memory to obtain SCG template data from the accelerometers and divide the obtained SCG template data into at least one cardiac cycle segment. The cardiac cycle segment is used to generate an SCG acceleration template which is in turn used to generate an SCG rotation matrix. SCG acceleration data is then obtained from the accelerometers and normalized by applying the generated SCG rotation matrix to the obtained SCG acceleration.

This application claims the benefit of priority of U.S. Provisional Application Ser. No. 62/635,183, filed on Feb. 26, 2018 the disclosure of which is herein incorporated by reference in its entirety.

### FIELD

This disclosure relates generally to wearable health devices and, more particularly, to a wearable health device system with compensated seismocardiography signals.

### BACKGROUND

Unless otherwise indicated herein, the materials described in this section are not prior art to the claims in this application and are not admitted as prior art by inclusion in this section.

Cardiovascular disease is among the leading causes of death. A number of diagnostic approaches have been developed to provide insight as to cardiovascular function in order to diagnose cardiovascular disease. These approaches include electrocardiography (ECG), echocardiography (ECHO), magnetic resonance imaging (MRI), and computerized tomography (CT) scan. These approaches focus on the physical structure of the heart and the electrical activities of the heart.

Another approach is auscultation which involves listening to the heart for audible sounds. Listening to the heart to detect potential issues is a developed skill which is useful in detecting certain structural issues which create unique sounds. By way of example, heart murmurs can be detected by auscultation.

An approach which differs fundamentally from the above described approaches is seismocardiography (SCG). Seismocardiography (SCG) is the detection/recording of body vibrations, typically at the sternum, which are induced by cardiovascular function. Based on these measurements, different parameters such as heart rate, heart rate variability, blood pressure estimation, cardiac output and also potential cardiovascular health problems can be identified. The information obtained through SCG may provide valuable diagnostic insight for ischemia detection, myocardial contractility, atrial fibrillation, and other cardiac issues. Because SCG is sensitive to vibrations, it can be used in diagnosing both mechanical and electrical issues related to cardiovascular function.

Unlike ballistocardiogram (BCG) techniques, which measure the forces of the body in reaction to the cardiac ejection of the blood, SCG utilizes wearable sensors such as accelerometers attached to the chest. Due to recent advancements in sensor technologies, SCG signals can be acquired with three dimensional (3D) accelerometers at a high sampling rate and bit resolution, which enables a detailed SCG evaluation. Thus, SCG evaluation is not subject to the limitation of simply summing acceleration based on cardiovascular forces (one dimension) as is the case for BCG methods.

The characteristics of the measured signals in SCG, however, are dependent on the measurement position (location and orientation of the sensor typically on the chest) and anatomical and physiological characteristics of the subject. Inter-subject variabilities are caused, e.g., by the variation in the position and orientation of the heart and the aorta between different subjects. The orientation of the various anatomical structures can vary between subjects by a number of degrees and be displaced by several centimeters. Intra-subject variability is primarily caused by translational and rotational errors after a sensor is removed and then reattached or replaced with another acceleration sensor on the chest of the same person. This results in a high inter- and intra-subject variability and makes a comparison between SCG signals difficult both between individuals and over a measurement period for a single individual. This is particularly problematic for automated evaluation routines.

Traditionally, precise orientation and location of internal anatomical structures is accomplished by invasive measures or by the use of complex imaging methods such as CT scans. The costs and inherent danger of invasive procedures as well as the cost of complex imaging procedures in order to reduce errors resulting from varying physiology inhibit wide usage of SCG sensors.

Accordingly, it would be beneficial if output from SCG sensor devices could be normalized so as to reduce errors/differences caused by sensor location and orientation. An automatic compensation would be further beneficial. A device which compensates for changes in sensor location and orientation which does not require invasive methods or complex imaging methods to establish orientation of internal anatomical structures would be further beneficial.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In accordance with one embodiment, a wearable health device system includes a housing configured to be worn by a subject, and a sensor assembly with at least two accelerometers which sense acceleration along non-parallel axes. A processor operably connected to the sensor assembly and a memory executes program instructions in the memory to obtain SCG template data from the accelerometers and divide the obtained SCG template data into at least one cardiac cycle segment. The cardiac cycle segment is used to generate an SCG acceleration template which is in turn used to generate an SCG rotation matrix. SCG acceleration data is then obtained from the accelerometers and normalized by applying the generated SCG rotation matrix to the obtained SCG acceleration data.

In one or more embodiments, the sensor assembly further includes a third accelerometer configured to sense acceleration along a third axis wherein the third axis is not parallel to the first axis or the second axis. The processor is further configured to execute the program instructions to obtain SCG template data from the third accelerometer, and obtain SCG acceleration data from the third accelerometer.

In one or more embodiments, the processor is configured to execute the program instructions to obtain SCG template data from the first and the second accelerometer by obtaining SCG data from two or more accelerometers at a 250 Hz rate for twenty seconds.

In one or more embodiments, the processor is configured to execute the program instructions to divide the obtained SCG template data into the at least one cardiac cycle segment using as a reference point at least one of a detected aortic valve opening peak and maximum force aorta (MFA) peak. The processor in some embodiments is configured to execute the program instructions to generate the SCG rotation matrix using a Nelder-Mead algorithm.

In one or more embodiments, the processor is configured to execute the program instructions to divide the obtained SCG template data into the at least one cardiac cycle segment using the MFA peak as a reference point, and to estimate a location of an aortic arch based upon the generated SCG rotation matrix.

In one or more embodiments, the processor is configured to execute the program instructions to divide the obtained SCG template data into the at least one cardiac cycle segment using electrocardiography (ECG) data as a reference point.

In one or more embodiments, the sensor assembly includes an ECG sensor configured to generate the ECG data. In some embodiments the sensor assembly also includes a gravity sensor.

In one or more embodiments, the processor is configured to execute the program instructions to generate the SCG rotation matrix using an Euler angles convention.

In accordance with one embodiment, a method of normalizing seismocardiography (SCG) data obtained with a wearable health device system includes positioning a wearable health device on a chest of a subject. Then, SCG template data is obtained from a first and a second accelerometer of a sensor assembly supported by a housing of the wearable health device by executing with a processor program instructions stored in a memory. The first accelerometer is configured to sense acceleration along a first axis, and the second accelerometer is configured to sense acceleration along a second axis which is not parallel to the first axis. The obtained SCG template data is divided into at least one cardiac cycle segment which is used to generate an SCG acceleration template. An SCG rotation matrix is generated using the generated SCG acceleration template and the SCG rotation matrix is applied to SCG acceleration data from the first accelerometer and the second accelerometer to generate normalized SCG acceleration data which is stored in the memory.

In one or more embodiment, another SCG template data is obtained from a third accelerometer of the sensor assembly by executing with the processor program instructions stored in the memory. The third accelerometer is configured to sense acceleration along a third axis, and the third axis is not parallel to the first axis or the second axis. SCG acceleration data is also obtained from the third accelerometer.

In one or more embodiment, the SCG data from the accelerometers is obtained at a 250 Hz rate for twenty seconds.

In one or more embodiment, the obtained SCG template data is divided into at least one cardiac cycle segment by dividing the obtained SCG template data into the at least one cardiac cycle segment using as a reference point at least one of a detected aortic valve opening peak and maximum force aorta (MFA) peak.

In one or more embodiment, the SCG rotation matrix is generated using a Nelder-Mead algorithm.

In one or more embodiment, the method further includes estimating a location of an aortic arch based upon the generated SCG rotation matrix when an MFA peak is used as a reference point.

In one or more embodiments, the obtained SCG template data is divided into at least one cardiac cycle segment by dividing the obtained SCG template data into the at least one cardiac cycle segment using electrocardiography (ECG) data as a reference point.

In one or more embodiments, the method includes generating the ECG data with an ECG sensor in the sensor assembly.

In one or more embodiments, generating with the processor an SCG rotation matrix includes generating the SCG rotation matrix using an Euler angles convention.

According to another aspect of the disclosure, a wearable health device includes a casing, and a sensing assembly configured to detect SCG signals. The sensing assembly is encapsulated in the casing, and a processor is communicatively coupled to the sensing assembly. The processor is configured to (a) acquire detected SCG signals, (b) divide the detected SCG signals into segments based on a reference point, (c) create an average acceleration template in two or more orientations, and (d) normalize the SCG signals. The processor can be either disposed in the casing or remotely located outside the sensing assembly. The wearable health device further comprises a computer readable medium communicatively coupled to the processor, the computer readable medium is configured to store SCG signals acquired by the processor. The computer readable medium is remotely located outside the wearable health device in some embodiments.

### BRIEF DESCRIPTION OF THE DRAWINGS

These and other features, aspects, and advantages of this disclosure will become better understood when the following detailed description of certain exemplary embodiments is read with reference to the accompanying drawings in which like characters represent like parts throughout the drawings, wherein:

FIG. 1 depicts a block diagram illustrating a wearable health device system in accordance with a described embodiment of the disclosure;

FIG. 2 depicts a plan schematic view of the wearable health device of FIG. 1 positioned on the chest of a subject;

FIG. 3 depicts simplified graphs of time-dependent SCG waveforms in two axes along with a temporally synchronized ECG waveform;

FIGS. 4-6 depict plan schematic views of the wearable health device of FIG. 1 positioned on the chest of a subject at −15°, −30°, and −45° rotations with respect to the depiction of FIG. 2;

FIG. 7 depicts simplified graphs of time-dependent SCG waveforms in three different axes as detected by the wearable health device of FIG. 1 when positioned as depicted in FIGS. 2, and 4-6 with the SCG signals temporally synchronized;

FIG. 8 depicts a process which is used in various embodiments to normalize detected SCG data from the wearable health device of FIG. 1;

FIGS. 9A and 9B depict different poses of an individual with the wearable health device of FIG. 1 attached to illustrate a sensor axes rotation correction that is optionally included in the process of FIG. 8;

FIG. 10A depicts a simplified graph of the effect of a two dimensional and three dimensional normalization of SCG data when the wearable health device is located and oriented nearly optimally on the chest of a subject showing minor modification of the SCG data by the normalization; and

FIG. 10B depicts a simplified graph of the effect of a two dimensional and three dimensional normalization of SCG data when the wearable health device is not located and oriented nearly optimally on the chest of a subject showing significant modification of the SCG data by the normalization.

### DETAILED DESCRIPTION

The following description is presented to enable any person skilled in the art to make and use the described embodiments, and is provided in the context of a particular application and its requirements. Various modifications to the described embodiments will be readily apparent to those skilled in the art, and the general principles defined herein may be applied to other embodiments and applications without departing from the spirit and scope of the described embodiments. Thus, the described embodiments are not limited to the embodiments shown, but are to be accorded the widest scope consistent with the principles and features disclosed herein.

Various operations may be described as multiple discrete actions or operations in turn, in a manner that is most helpful in understanding the claimed subject matter. However, the order of description should not be construed as to imply that these operations are necessarily order dependent. In particular, these operations need not be performed in the order of presentation. Operations described may be performed in a different order than the described embodiment. Various additional operations may be performed and/or described operations may be omitted in additional embodiments.

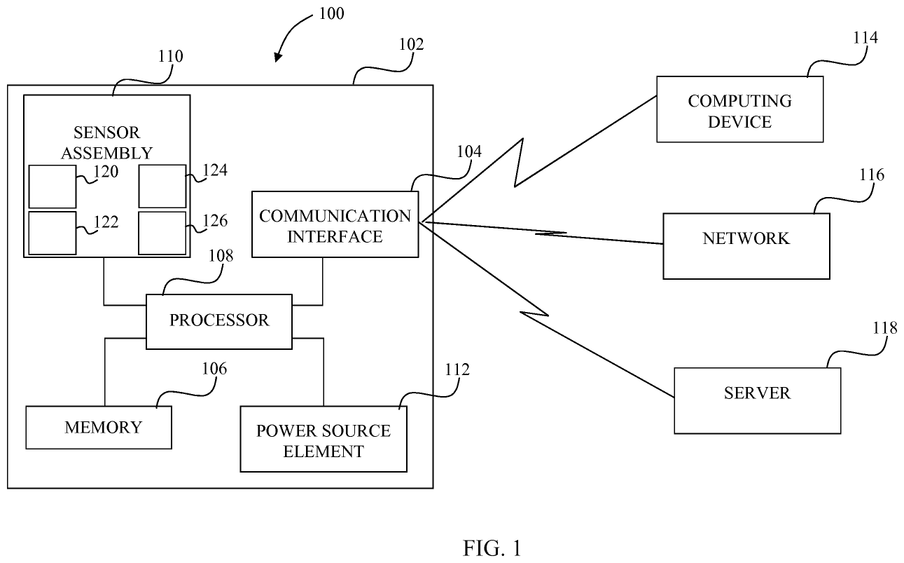

FIG. 1 illustrates a simplified block diagram of a wearable health device 100 configured to be worn by a subject, e.g. a user, a patient, or a test subject. In use, the wearable device 100 is attached or applied to the subject's body. The wearable device 100, in one embodiment, is a patch. The wearable health device 100 includes a housing 102 which encapsulates the components of the wearable health device 100 and a suitable adhesive such as a bio-compatible double sided tape on one side or one surface of the housing 102.

As illustrated in FIG. 1, the components encapsulated in the housing of the wearable device 100 in one embodiment include a communication unit or a communication interface 104, a memory or a machine-readable medium 106, a processor or a processing unit 108, and a sensor assembly 110. In some embodiments, the wearable health device 100 includes other computer implemented modules suitable for the desired application. The computer implemented modules in one or more embodiments include an input user interface, a display, an antenna, and so forth. The wearable health device 100 is powered by a power-source element or an energy storage element 112.

The communication unit 104 forms one or more links with external computing devices 114, networks 116, and/or servers 118 so as to transfer software, data, public key, private key, and/or communication protocol between the wearable health device 100 and the devices 114, networks 116, and/or servers 118. The link is established in one or more embodiments wirelessly, by a wired communication path, and combinations thereof.

The machine 114 in different embodiments is one or more of smartphones, tablets, laptops, computers, phablets, personal digital assistants (PDAs), cellphones, voice-controlled devices such as Echo, Alexa, homepod, and the like. The network 116 in various embodiments is one or more of cloud networks, PSTNs, WANs, WLANs, and so forth.

The software, data, public key, private key, and/or communication protocol transferred to or obtained by the wearable health device 100 is stored within the memory 106. The memory 106 is a transitory machine-readable medium, non-transitory machine-readable medium, volatile machine-readable medium, non-volatile machine-readable medium, magnetic machine-readable medium, optical machine-readable medium, or electrical media, such as a random access memory (RAM), read-only memory (ROM), non-volatile RAM (NVRAM), electrically-erasable programmable ROM (EEPROM), flash memory, or any other digital or analog media.

The processor 108 in different embodiments includes one or more levels of caching, such as a level cache memory, one or more processor cores, and registers. In various embodiments the processor 108 is a microprocessor (0), a microcontroller (μC), a digital signal processor (DSP), and any combination thereof. The exemplary processor cores may (each) include an arithmetic logic unit (ALU), a floating point unit (FPU), a digital signal processing core (DSP Core), or any combination thereof. A memory controller is used with the processor 108 in some embodiments. In some embodiments the memory controller is an internal part of the processor 108. The processor 108 is configured to execute program instructions stored in the memory 106.

Program or computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, objects, components, and data structures, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The energy storage element 112 in various embodiments is configured for inductive charging, qi charging, energy harvesting, wired charging, wireless charging, and any suitable charging method for transferring power to the wearable health device 100.

The sensor assembly 110 includes at least one sensor and in the embodiments depicted includes four sensors 120, 122, 124, and 126. The sensors 120, 122, 124, and 126 in different embodiments include one or more of single axis accelerometers, double-axis accelerometers, tri-axis accelerometers, gyroscopes, orientation sensors, rotation sensors, microphones, gravity sensors, ECG sensors, and so forth.

Each embodiment includes a sensor or sensors sufficient to provide acceleration sensing in at least two axes. Thus, in one embodiment, the sensors 120 and 122 are single axis accelerometers. In another embodiment, the sensor 124 is a double axis accelerometer. In one embodiment, the sensor 126 is a multi-axis accelerometer in the form of a tri-axis accelerometer. In one embodiment, the tri-axis accelerometer is model number BMA280 commercially available through Robert Bosch Sensortec of Mount Prospect, Ill., USA.

Although one sensor assembly 110 is illustrated in the embodiment of FIG. 1, in other embodiments more than one sensor assembly is incorporated in the wearable health device 100 to detect and/or measure one or more parameters associated with either contractile properties of the subject's heart or the subject's blood flow.

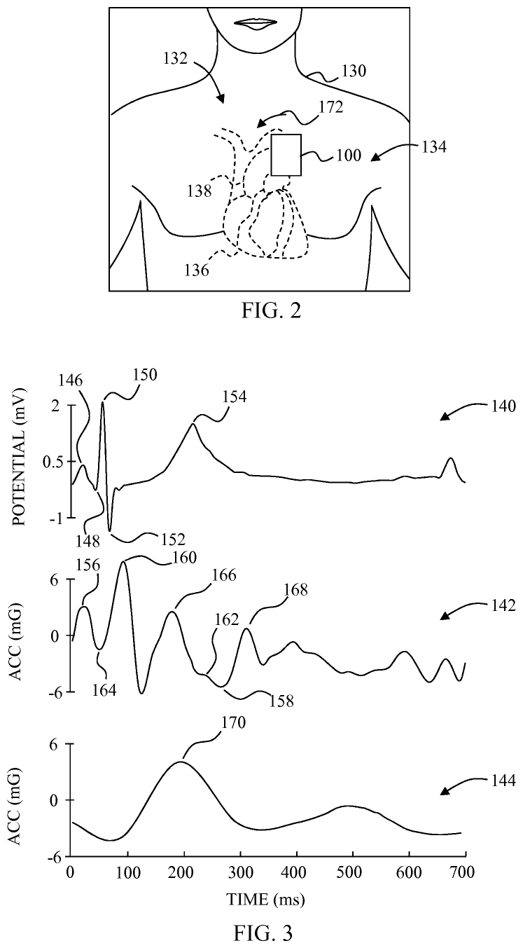

FIG. 2 illustrates a subject 130, such as a user or a patient, which in this embodiment is a human, wearing the wearable health device 100. The wearable health device 100 in one embodiment measures the mass transit time (MTT) and/or the pulse transit time (PTT), and monitors vital signs as described in detail in U.S. application Ser. No. 15/564,585 entitled “Blood Pressure and Cardiac Monitoring System and Method”, U.S. Appl. Ser. No. 62/583,754 entitled “Vital Signs Monitoring System and Method”, and PCT Appl. Ser. No. [ATTY docket no. 1576-2271PCT] filed the same day herewith and entitled “Wearable Health Device System With Automatic Referencing of Seismocardiography Signals” which claims priority to U.S. Appl. Ser. No. 62/635,824 entitled “Angle Errors Compensation Methods and Systems for Wearable Health Devices”, the contents of which are incorporated by reference.

As illustrated in FIG. 2, the wearable health device 100 is positioned on the chest 132 of the subject 130 at a location which is to the left of the subject's sternum (not shown for clarity sake, but located directly beneath the chin of the subject) at the upper portion of the subject's breast 134. The wearable health device 100 is positioned in one embodiment by removing or peeling off a cover from the adhesive surface of the patch before attaching the adhesive surface directly to the body of the subject 130.

At the location depicted in FIG. 2, the wearable health device 100 is typically located slightly above the heart 136 and directly over a portion of the aortic arch 138. As noted above, the actual anatomy of the individual typically varies from the depicted anatomy, and the position of the wearable health device 100 on a particular subject will also vary. When the wearable health device 100 is positioned in the manner depicted in FIG. 2, the conventional frame of reference, and the frame of reference used herein and in the claims unless otherwise explicitly stated, is centered on the device 100 with the x-axis extending vertically, the y-axis extending horizontally, and the z-axis extending into/out of the page through the center of the wearable health device 100.

The wearable health device 100, when activated, either manually or automatically, acquires seismocardiography (SCG) data noninvasively and continuously with a maximum of comfort and ease. FIG. 3 depicts exemplary data obtained during a single cardiac cycle from the wearable health device 100. In this embodiment, the sensor assembly includes an electrocardiogram (ECG) sensor, z-axis accelerometer, and an x-axis accelerometer. Accordingly, FIG. 3 depicts ECG data 140, z-axis data 142, and x-axis data 144. The data are temporally aligned.

Multiple local minima and maxima are discernable from FIG. 3 which provide insight as to physiological events during the cardiac cycle. The ECG data 140 shows the “P wave” 146, the “QRS complex” including the “Q wave” 148, the “R wave” 150, and the “S wave” 152. The ECG data further includes the “T wave” 154.

The z-axis data 142 reflects mitral valve closing (MC) 156, mitral valve opening (MO) 158, aortic valve opening (AO) 160, aortic valve closing (AC) 162, isovolumetric contraction (IVC) 164, rapid ejection (RE) 166, and rapid filling (RF) 168. The x-axis data 144 reflects maximum blood flow through the aortic arc which is referred to as maximum force aorta (MFA) 170.

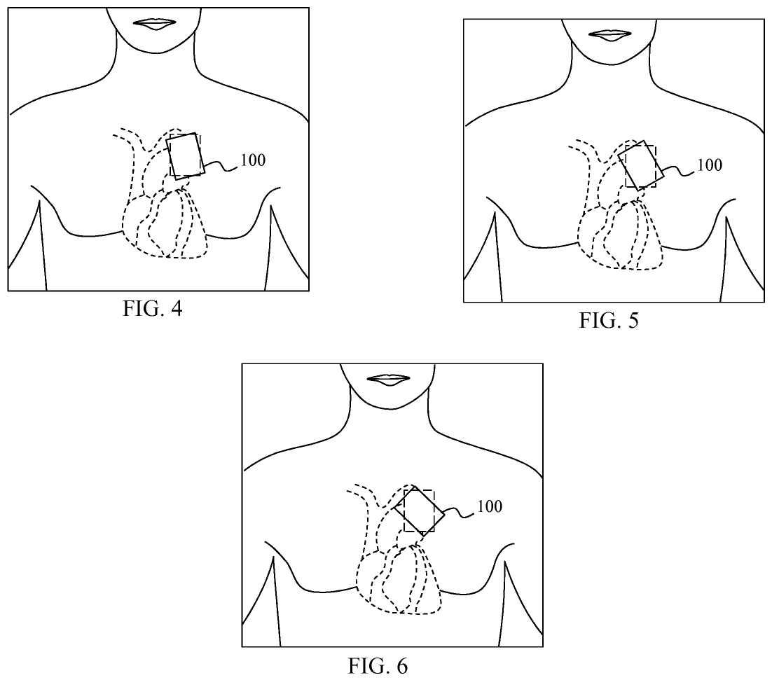

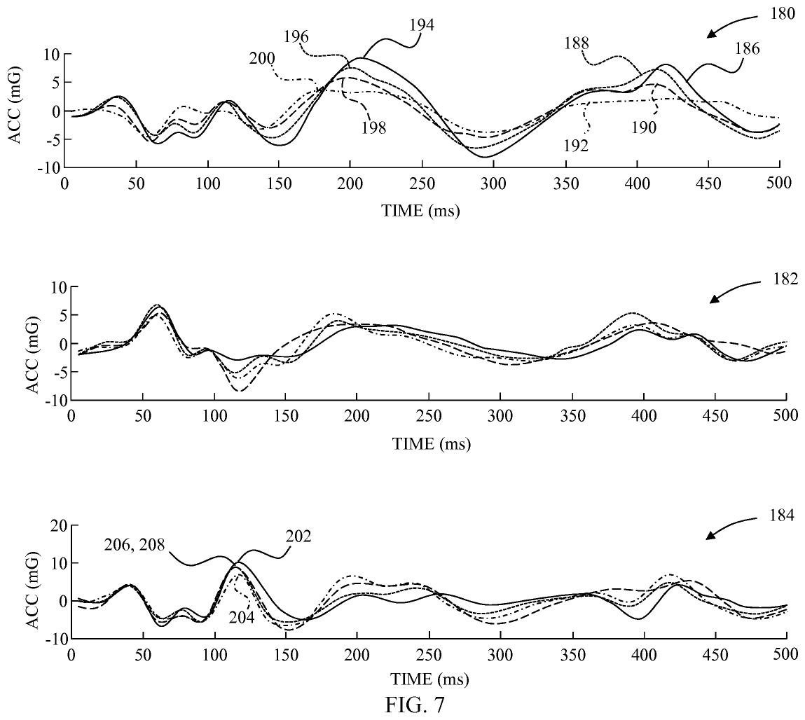

The prominence of the minima and maxima depends on the sensor position and orientation as well as on the anatomy of the subject. By way of example, after obtaining data with the wearable health device 100 in the position depicted in FIG. 2, the wearable health device 100 was rotated in the direction of the arrow 172 in FIG. 2 by about −15° to the position depicted in FIG. 4 and additional data was obtained. The process was repeated with the wearable health device rotated by −30° (FIG. 5) and −45° (FIG. 6). The original position of the wearable health device 100 is depicted in shadow in FIGS. 4-6. For every location, the z-axis was maintained so that the wearable health device 100 was rotated in each step by 15° about the z-axis in the negative direction. FIG. 7 depicts the data obtained from the tri-axis accelerometer 126.

FIG. 7 depicts x-axis data 180, y-axis data 182, and z-axis data 184. Each data was obtained at each of the four positions depicted in FIGS. 2 and 4-6. Thus, x-axis data 180 includes 0° rotational data 186, −15° rotational data 188, −30° rotational data 190, and −45° rotational data 192. FIG. 7 shows that as the wearable health device 100 was rotated the amplitude of the MFA peak decreased and timing of the MFA peak occurred earlier from the 0° MFA 194, to the −15° MFA 196, to the −30° MFA 198 to the −45° MFA 200.

The peak associated with the AO also showed variability as evidenced by the z-axis data 184. The amplitude of the AO peak was lowered from the 0° AO 202 to the later (temporally) −45° AO 204 while the −15° AO 206 and the −30° AO 208 amplitudes were between the 0° AO 202 and the −45° AO 204, while occurring earlier in the pattern.

The results of FIG. 7 illustrate that the orientation of the wearable health device 100 results in changes in the data observed. Likewise, movement of the wearable axis along either or both of the x-axis and the y-axis (i.e. repositioning of the z-axis) will result in amplitude and temporal changes in the observed data.

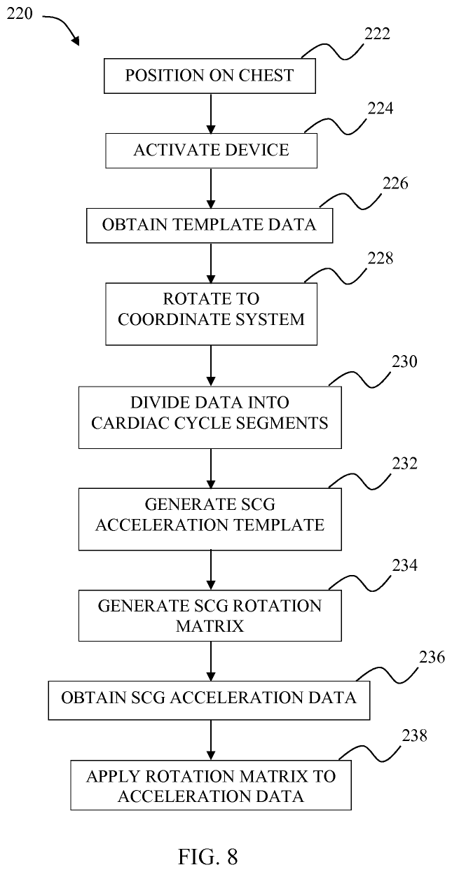

Accordingly, the wearable health device 100 is configured to perform the method 220 of FIG. 8 to compensate for orientation and location differences. At block 222 the wearable health device 100 is positioned on the chest of the subject. In some embodiments attaching the wearable health device 100 (which may be a patch) onto the body of the subject includes removing or peeling a cover from an adhesive surface of the path and attaching the adhesive surface directly to the body of the subject. The wearable health device 100 is optimally positioned at the location and orientation depicted in FIG.

At block 224 the wearable health device 100 is activated either manually or automatically, and SCG template data is acquired by the sensor assembly 110 (block 226). “SCG template data” is SCG data acquired using one or more sensors configured to obtain data for at least two non-parallel axes, and preferably at least three axes. The data is acquired over a period of time sufficient to obtain at least one cardiac cycle, and preferably multiple cardiac cycles and with a frequency sufficient to characterize the local maxima and minima, and stored in the memory 106. In accordance with one embodiment, the data is acquired over a period of at least 20 seconds at a frequency of at least 250 Hz.

In some embodiments, the sensor coordinate system is then rotated into a normative coordinate system at block 228 to correct for the manner in which the wearable health device 100 lays on the subject. Rotation of the coordinate system facilitates annotation of the SCG which is discussed below.

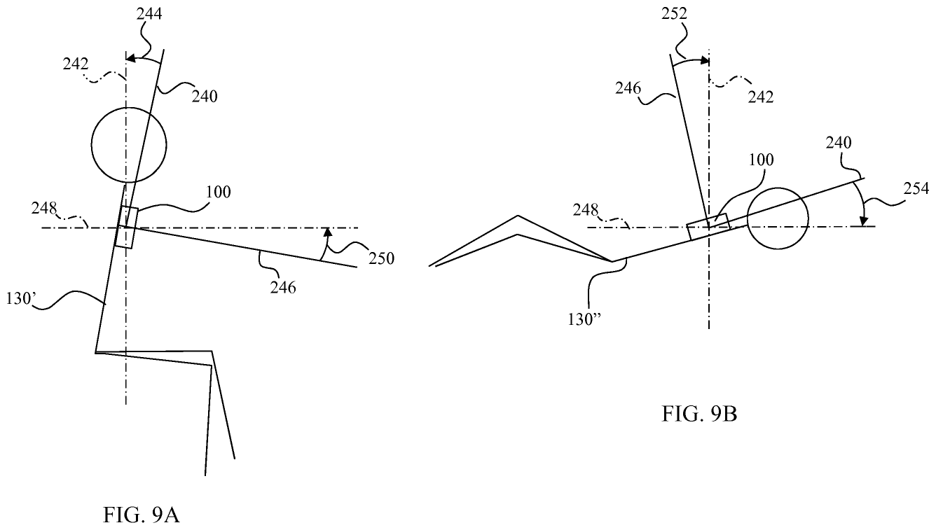

The rotation to correct for the positioning of the sensor is determined based upon gravity and the general orientation of the wearable health device 100. An example of this rotation for a three-axes device is explained with reference to FIGS. 9A and 9B. A subject will either be sitting, standing, or laying down when initial data is acquired. In both a sitting and standing position, the wearable health device 100 will have the general orientation shown with respect to the subject 130′ in FIG. 9A. In this position, the x-axis 240 of the wearable health device 100 is close to the gravity axis 242. The angle of rotation 244 for the x-axis is thus determined using either a gravity sensor in the sensor assembly 110 or an external instrument so as to align the rotated x-axis of the wearable health device 100 with gravity 242. The z-axis 246 of the wearable health device 100 is also modified to a rotated z-axis 248 by an identical angle of rotation 250.

A similar rotation occurs when the subject is laying down. In this scenario depicted with the subject 130″ of FIG. 9B, however, the z-axis 246 of the wearable health device 100 is closest to the gravity axis 242. The angle of rotation 252 for rotation of the z-axis 246 is thus determined using either a gravity sensor in the sensor assembly 110 or an external instrument to align the rotated z-axis with gravity 242. The x-axis 240 of the wearable health device 100 is also modified to a rotated x-axis 248 by an identical angle of rotation 254.

In both scenarios depicted in FIGS. 9A and 9B, the y-axis of the wearable health device 100 is assumed to be perpendicular to the gravity axis 242. Preferably, this is ensured by proper positioning of the subject prior to obtaining the data in block 224. With the known angles of rotation, along with the knowledge of which axis was rotated to align with the gravity axis 242, the obtained data can be converted to any desired world coordinate system. In embodiments incorporating attitude sensors in the sensor assembly 110, the y-axis in some embodiments is corrected based upon the gravity axis. In some embodiments, the sensor axes rotation data is stored in the memory 106. In other embodiments, the sensor axes rotation data is stored in a remote memory such as a memory associated with the computing device 114, the network 116, or the server 118, and applied to data received from the wearable sensor device 100.

Returning to FIG. 8, the obtained data is divided into cardiac cycle segments with each cardiac cycle segment including a single cardiac cycle (block 230). The segmentation is based upon any desired reference point. Accordingly, in some embodiments, the reference points are determined by characteristic SCG points while in other embodiments external signals which are temporally synchronized with the SCG data are used either additionally or in place of a characteristic SCG point.

In some embodiments, the SCG based reference point is the timing of the opening of the aortic flaps using the AO peak 160 of FIG. 3. The AO peak 160 is detected/determined manually in some embodiments whereas in other embodiments it is detected/determined automatically, e.g., by the wearable device 100. External reference points in different embodiments are generated by various sensor signals such as ECG (e.g. detection of R-peak) or acoustic signals.

At block 232 an SCG acceleration template is generated using the cardiac cycle segments. Initially, the cardiac cycle segments are interpolated to a unit length and the arithmetic average is aggregated to provide an average cardiac cycle segment. The average cardiac cycle segment includes all of the data for the two or more, preferably three, axes of the accelerometers of the wearable health device 100.

The average cardiac cycle segment is then rotated to identify the orientation in three dimensional space at which the selected reference point is at a maximum in the associated axis, typically the x-axis or z-axis. Rotation of the average cardiac cycle segment to generate a rotation matrix is accomplished in various embodiments by numerical methods like the Nelder-Mead algorithm.

In some embodiments, the optimal rotation matrix is estimated by transforming the sensor data from a Cartesian coordinate system into a polar coordinate system (transformation of the data from x/y/z axes into amplitude/angle representation). One such embodiment is described in more detail in PCT Appl. Ser. No. [ATTY docket No. 1576-2271PCT] filed the same day herewith and entitled “Wearable Health Device System With Automatic Referencing of Seismocardiography Signals.” Based on these angle values a rotation matrix is computed in one embodiment by the processor 108 using an Euler angles convention.

In any event, an SCG rotation matrix is generated (block 234) and stored. The SCG rotation matrix, which in some embodiments incorporates the sensor axes rotation data, is stored in the memory 106. In some embodiments, more than one rotation matrix is generated for a given set of data so as to optimize the normalized data to different reference points. In some embodiments, the rotation matrix is stored in a remote memory such as a memory associated with the computing device 114, the network 116, or the server 118, and applied to data received from the wearable sensor device 100.

The wearable health device 100 is then used to collect SCG acceleration data at block 236. SCG acceleration data is SCG data acquired using at least the one or more sensors used to obtain SCG template data. In some embodiments, collection of the SCG acceleration data is accomplished prior to block 228, or at any other desired time including before block 226. The SCG acceleration data is stored in the memory 106 or transmitted in real time or near real time to one or more of the computing device 114, the network 116, or the server 118. The SCG acceleration data typically includes a substantially larger amount of data than the SCG template data and can include the SCG template data.

At block 238 the SCG rotation matrix, which optionally includes the sensor axes rotation, is applied to the collected SCG acceleration data to generate normalized SCG acceleration data. In some embodiments the SCG rotation matrix, and optionally the sensor axes rotation, is applied prior to storing the data. The normalized SCG acceleration data in some embodiments is provided in a database with other normalized SCG acceleration data. Because the data has been normalized, more accurate comparisons can be made since sensor placement errors (orientation of the accelerometers) and anatomical variations between subjects (orientation of the aortic arch) are accounted for. The normalization method is generic and can be used in different applications.

Moreover, the normalized SCG data is used in some embodiments to estimate the position and orientation of certain anatomical structures such as the aortic arc. Specifically, a rotation vector is computed based on the generated SCG acceleration template. The rotation vector points toward the location of the anatomical reference point. Accordingly, by generating the SCG acceleration template using the MFA as the selected reference point, the rotation vector for the peak points toward the aortic arc. If the position and orientation of the sensor on the chest is known, the orientation of the anatomic reference structure can be estimated. The accuracy of this procedure can be further improved by the acquisition of SCG data at different chest positions.

Thus, the disclosed method can be further used to estimate the orientation of anatomical structures (e.g. aortic arc). In contrast to expensive imaging techniques (e.g. MRT), the disclosed method is inexpensive and can be performed outside hospital environments.

The disclosed embodiments thus provide SCG data which can be easily obtained while increasing the precision in comparison between data collections. The SCG data can be obtained without the need for expensive procedures.

Moreover, the SCG data can be obtained without a subject ever going to a health provider. A wearable health device with or without a gravity sensor in the sensor assembly can be purchased at, e.g., a local pharmacy or otherwise delivered to a subject. The device is then positioned by the subject or an individual on the subject's chest. The wearable health device then optionally ascertains the gravity axis as described above, and stores that data along with the acceleration data. At the end of the prescribed data collection duration, the wearable health device is removed and sent to a remote facility where the desired remaining steps of the method of FIG. 8 are performed.

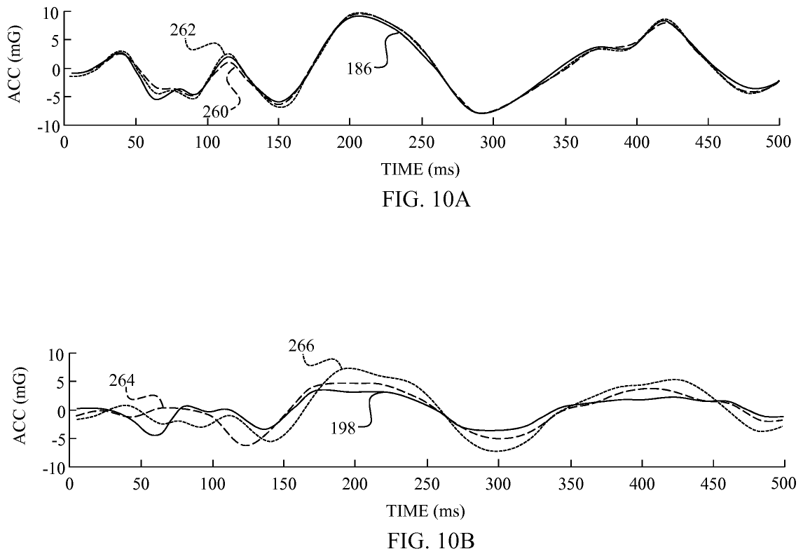

The foregoing process was verified using the SCG data from FIG. 7. FIG. 10A depicts the 0° rotation data 186 from the x-axis data 144 of FIG. 7. FIG. 10A further depicts two axis normalized SCG data 260 generated by applying a two axis SCG rotation matrix to the 0° rotation data 186. Also depicted is three axis normalized SCG data 262 generated by applying a three axis SCG rotation matrix to the 0° rotation data 186. As is evident from FIG. 10A, the original 0° rotation data 186 is reasonably accurate, indicating that the wearable sensor device 100 was at a close to optimal location.

FIG. 10B depicts the 45° rotation data 198 from the x-axis data 144 of FIG. 7. FIG. 10B further depicts two axis normalized SCG data 264 generated by applying a two axis SCG rotation matrix to the −45° rotation data 198. Also depicted is three axis normalized SCG data 266 generated by applying a three axis SCG rotation matrix to the −45° rotation data 198. As is evident from FIG. 10B, the original −45° rotation data 198 significantly modified by the SCG rotation matrices, indicating that the wearable sensor device 100 was not at a close to optimal location. FIG. 10B further shows that the actual peak is not only larger, but is also transposed temporally along the axis by about 20 ms.

The system and method described above reduces intra-subject variability of SCG data which occurs when a single subject performs multiple SCG measurements with frequent manual attachment and detachment of the sensor setup. The SCG data in this scenario is adversely affected by placement errors of the sensor setup (position and orientation of the setup will not be the same each time).

The disclosed system and method further reduces inter-subject variability of SCG data which occurs when SCG data is acquired across multiple subjects. In this scenario the SCG data has a high variability due to anatomical differences between subjects as well as difference in placement on the various subjects.

The disclosed embodiments are thus useful for many different use-cases. Examples include long term monitoring of hypertonia patients, sleep monitoring, and monitoring of subjects with cardiovascular diseases. In addition comparisons between different subjects is improved and automated evaluation systems can be used.

While the disclosure has been described with reference to various embodiments, it will be understood that these embodiments are illustrative and that the scope of the disclosure is not limited to them. Many variations, modifications, additions, and improvements are possible. More generally, embodiments in accordance with the patent have been described in the context or particular embodiments. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A wearable health device system comprising:
a housing configured to be worn by a subject
a sensor assembly supported by the housing and including a first accelerometer configured to sense acceleration along a first axis, and a second accelerometer configured to sense acceleration along a second axis which is not parallel to the first axis;
a memory including program instructions stored therein; and
a processor operably connected to the sensor assembly and the memory, the processor configured to execute the program instructions to
obtain seismocardiography (SCG) data from the first and the second accelerometer,
divide the obtained SCG data into at least one cardiac cycle segment,
generate an SCG acceleration template using the at least one cardiac cycle segment,
generate an SCG rotation matrix using the generated SCG acceleration template,
obtain SCG acceleration data from the first accelerometer and the second accelerometer,
generate normalized SCG acceleration data by applying the generated SCG rotation matrix to the obtained SCG acceleration data, and
store the normalized SCG acceleration data.

2. The wearable health device system of claim 1, wherein:
the sensor assembly further includes a third accelerometer configured to sense acceleration along a third axis;
the third axis is not parallel to the first axis or the second axis; and
the processor is further configured to execute the program instructions to
obtain SCG template data from the third accelerometer, and
obtain SCG acceleration data from the third accelerometer.

3. The wearable health device system of claim 2, wherein:
the processor is configured to execute the program instructions to obtain SCG template data from the first the second, and the third accelerometer by obtaining SCG data from the first, the second, and the third accelerometer at a 250 Hz rate for twenty seconds;
the processor is configured to execute the program instructions to divide the obtained SCG template data into the at least one cardiac cycle segment using as a reference point at least one of a detected aortic valve opening peak and maximum force aorta (MFA) peak; and
the processor is configured to execute the program instructions to generate the SCG rotation matrix using a Nelder-Mead algorithm.

4. The wearable health device system of claim 1, wherein the processor is configured to execute the program instructions to divide the obtained SCG template data into the at least one cardiac cycle segment using as a reference point at least one of a detected aortic valve opening peak and maximum force aorta (MFA) peak.

5. The wearable health device system of claim 1 wherein:
the processor is configured to execute the program instructions to divide the obtained SCG template data into the at least one cardiac cycle segment using the MFA peak as a reference point; and
the processor is further configured to execute the program instructions to estimate a location of an aortic arch based upon the generated SCG rotation matrix.

6. The wearable health device system of claim 1, wherein the processor is configured to execute the program instructions to divide the obtained SCG template data into the at least one cardiac cycle segment using electrocardiography (ECG) data as a reference point.

7. The wearable health device system of claim 6, wherein the sensor assembly includes an ECG sensor configured to generate the ECG data.

8. The wearable health device system of claim 1, wherein the processor is configured to execute the program instructions to generate the SCG rotation matrix using a Nelder-Mead algorithm.

9. The wearable health device system of claim 1, wherein the processor is configured to execute the program instructions to generate the SCG rotation matrix using an Euler angles convention.

10. The wearable health device system of claim 1, wherein the processor is configured to execute the program instructions to obtain SCG template data from the first and the second accelerometer by obtaining SCG data from the first and the second accelerometer at a 250 Hz rate for twenty seconds.

11. A method of normalizing seismocardiography (SCG) data obtained with a wearable health device system comprising:
positioning a wearable health device on a chest of a subject;
obtaining SCG template data from a first and a second accelerometer of a sensor assembly supported by a housing of the wearable health device by executing with a processor program instructions stored in a memory, wherein the first accelerometer is configured to sense acceleration along a first axis, and the second accelerometer is configured to sense acceleration along a second axis which is not parallel to the first axis;
dividing with the processor the obtained SCG template data into at least one cardiac cycle segment;
generating with the processor an SCG acceleration template using the at least one cardiac cycle segment;
generating with the processor an SCG rotation matrix using the generated SCG acceleration template;
obtaining with the processor SCG acceleration data from the first accelerometer and the second accelerometer;
generating normalized SCG acceleration data by applying the generated SCG rotation matrix to the obtained SCG acceleration data; and
storing the normalized SCG acceleration data in the memory.

12. The method of claim 11, further comprising:
obtaining SCG template data from a third accelerometer of the sensor assembly by executing with the processor program instructions stored in the memory, wherein the third accelerometer is configured to sense acceleration along a third axis, and the third axis is not parallel to the first axis or the second axis; and
obtaining with the processor SCG acceleration data from the third accelerometer.

13. The method of claim 12, wherein:
obtaining SCG template data comprises obtaining SCG data from the first, the second, and the third accelerometer at a 250 Hz rate for twenty seconds;
dividing with the processor the obtained SCG template data into at least one cardiac cycle segment comprises dividing the obtained SCG template data into the at least one cardiac cycle segment using as a reference point at least one of a detected aortic valve opening peak and maximum force aorta (MFA) peak; and
generating with the processor an SCG rotation matrix comprises generating the SCG rotation matrix using a Nelder-Mead algorithm.

14. The method of claim 11, wherein dividing with the processor the obtained SCG template data into at least one cardiac cycle segment comprises dividing the obtained SCG template data into the at least one cardiac cycle segment using as a reference point at least one of a detected aortic valve opening peak and maximum force aorta (MFA) peak.

15. The method of claim 11 wherein:
dividing with the processor the obtained SCG template data into at least one cardiac cycle segment comprises dividing the obtained SCG template data into the at least one cardiac cycle segment using the MFA peak as a reference point, the method further comprising:
estimating with the processor a location of an aortic arch based upon the generated SCG rotation matrix.

16. The method of claim 11, wherein dividing with the processor the obtained SCG template data into at least one cardiac cycle segment comprises dividing the obtained SCG template data into the at least one cardiac cycle segment using electrocardiography (ECG) data as a reference point.

17. The method of claim 16, further comprising:
generating the ECG data with and ECG sensor in the sensor assembly.

18. The method of claim 11, wherein generating with the processor an SCG rotation matrix comprises generating the SCG rotation matrix using a Nelder-Mead algorithm.

19. The method of claim 11, wherein generating with the processor an SCG rotation matrix comprises generating the SCG rotation matrix using an Euler angles convention.

20. The method of claim 11, wherein obtaining SCG template data comprises obtaining SCG data from the first and the second accelerometer at a 250 Hz rate for twenty seconds.
